(19) United States
(12) Patent Application Publication
(10) Pub. No.: US 2019/0208754 A1
(43) Pub. Date: Jul. 11, 2019

(54) METHOD OF CONSTRUCTING HUMANIZED MURINE MODEL OF CHRONIC VIRAL HEPATITIS USING STEM CELL

(71) Applicant: Zhejiang University, Hangzhou (CN)

(72) Inventors: Jun Li, Hangzhou (CN); Suwan Sun, Hangzhou (CN); Jiang Li, Hangzhou (CN); Jing Jiang, Hangzhou (CN)

(21) Appl. No.: 16/355,707

(22) Filed: Mar. 16, 2019

Related U.S. Application Data
(63) Continuation-in-part of application No. PCT/CN2017/081750, filed on Apr. 24, 2017.

(30) Foreign Application Priority Data
Mar. 7, 2017 (CN) 201710130522.7

(51) Int. Cl. A01K 67/027; A61K 9/00; C12N 5/0735

(52) U.S. Cl. CPC A01K 67/0278; A61K 9/0053; A61K 9/0019; A01K 2207/12; A01K 67/0271; A01K 2227/105; A01K 2267/0337; C12N 5/0606

(57) ABSTRACT

A method of constructing humanized murine model using stem cells, includes obtaining human stem cells; transplanting human stem cells into murine with liver damage; and obtaining hepatotropic virus infected humanized murine. It was found that by inducing severe liver damage and transplanting human stem cells, human-derived hepatocytes in murine liver have a high chimeric rate of 50-95%, and human-derived immune cells may exist in murine organs such as spleen, blood, liver, and bone marrow, thereby forming murine model of humanized liver and immune cell. The humanized murines are then infected with various types of hepatotropic viruses to form humanized hepatotropic viral infected murine model. In addition to construction of model for studying hepatotropic viral infection using the technique for constructing the humanized murine model, the concept of this technical solution may also be used for constructing models of other humanized organs.

### CROSS REFERENCE TO RELATED APPLICATIONS

This application is a continuation-in-part application of PCT Application No. PCT/CN2017/081750 filed on Apr. 24, 2017, which claims priority to Chinese Patent Application No. 201710130522.7 filed on Mar. 7, 2017, the entire contents of which are incorporated herein by reference.

### FIELD OF TECHNOLOGY

The present invention relates to the fields of clinical medicine, experimental medicine, regenerative medicine and virology, and specifically relates to a method of constructing a humanized murine model for studying human diseases such as viral hepatitis.

### BACKGROUND

Hepatotropic viruses (hepatitis A virus (HAV), hepatitis B virus (HBV), hepatitis C virus (HCV), etc.) have a wide prevalence and millions of people die from liver failure, liver cirrhosis and hepatocellular carcinoma caused by viral hepatitis every year. Hepatotropic viruses can only cause diseases in high-level primates. The use of apes is often required in the animal testing models. Experiment with apes has a high cost, complicated operation, and long test cycle, which seriously hinder the research on the mechanism, progress, and outcome of viral hepatitis and greatly limit the optimization of treatment options. Therefore, it is of great scientific significance and application value to establish small animal models which are susceptible to hepatotropic viral infection and diseases. The establishment of a humanized murine model will provide a good research vehicle for the study of viral hepatitis, and the treatment and disease outcome thereof. Stem cells (including human embryonic stem cells, mesenchymal stem cells, induced pluripotent stem cells, etc.) have the ability of multipotent differentiation, which can differentiate into various types of functional cell. In our previous study, it was found that human bone marrow mesenchymal stem cells (hBMSCs) transplantation rescued fulminant hepatic failure (FHF) in pigs and there were no immunological rejections occurred. This laid the foundation for us to transplant stem cells into the damaged liver of the murine and establish a new type of humanized chimeric murine model. This humanized murine provides a safeguard for elucidating the biological characteristics of hepatotropic virus, the pathogenesis of hepatitis, the disease outcome, and for developing and screening the new anti-hepatotropic viral drugs. Using medical techniques of stem cell transplantation and liver regeneration to establish an animal model that is highly similar to human hepatotropic viral infection and can mimic the natural progression of hepatotropic virus, this will provide an excellent animal experiment platform for studying the pathogenesis of hepadnavirus, developing new anti-hepatitis virus drugs, and evaluating the effectiveness of the relevant vaccines.

### SUMMARY OF THE INVENTION

In view of the deficiency of the existing models for studying hepatotropic virus, the present invention provides a technique for constructing a humanized murine model using human stem cells. The present invention is realized by the following technical solutions:

The present invention discloses a method of constructing a humanized murine model using human stem cells, including the following steps:
(a) obtaining the human stem cells;
(b) transplanting the human stem cells into a murine with liver damage; and
(c) obtaining a hepatotropic virus infected humanized murine.

In a preferred embodiment, the human stem cells are isolated and cultured human stem cells, or commercialized isolated or frozen human stem cells or cell line.

In a preferred embodiment, the isolated and cultured human stem cells are obtained by the following steps:
(i) obtaining purified human stem cells;
(ii) culturing and subculturing the purified human stem cells; and
(iii) incubating the cultured, subcultured, purified human stem cells obtained from the step
(ii) in an incubator at 20° C. to 40° C., 2% to 10% CO₂.

In a preferred embodiment, the step (b) includes the following steps:

(b1) obtaining different strains of test murine;
(b2) establishing the murine with liver damage by administering a liver-damaging drug, or applying a partial hepatic resection by surgery; and
(b3) transplanting 1×10⁴⁻⁸ of the human stem cells into the murine with liver damage.

In a preferred embodiment, the step (b) further includes the following step after the step (b3):
(b4) administering the liver-damaging drug in several times.

In a preferred embodiment, the test murine is selected from the group consisting of normal mouse, immunodeficient mouse, normal rat, and immunodeficient rat; wherein the liver damage includes any one of the following: acute liver damage, chronic liver damage, acute liver failure, subacute liver failure, and chronic liver failure.

In a preferred embodiment, in the step (b2), the liver-damaging drug is administered through intraperitoneal injection, intramuscular injection, peripheral intravenous injection, oral administration, or gastric administration.

In a preferred embodiment, in the step (b3), the 1×10⁴⁻⁸ of the human stem cells are transplanted through peripheral intravenous injection, portal vein injection, spleen injection, or liver injection.

In a preferred embodiment, the step (c) includes injecting each of the murine with liver damage with various types of hepatotropic viruses through peripheral intravenous injection, subcutaneous injection, intramuscular injection, or intraperitoneal injection.

In a preferred embodiment, the method further includes the following step after the step (c):
(d) measuring viral load once within 3-30 days after the murine with liver damage is infected to confirm that the humanized murine model is successfully established; after measuring the viral load for once within 3-30 days after the murine with liver damage is infected, measuring the viral load again thereafter in several times to confirm that the humanized murine model is successfully established.

Comparing to the existing technique for constructing humanized murine models, the advantageous effects of the present invention are as follows:

The human stem cells (such as human mesenchymal stem cells, human embryonic stem cells, and induced pluripotent stem cells) used are negative for surface antigens such as CD34 and CD45 and positive for surface antigens such as CD29 and CD90, and can be simultaneously differentiated into human hepatocytes and immune cells. A liver and immune cell dual-humanized murine model can be constructed simply by transplanting a single type of stem cell. However, the existing humanized models were developed through the co-transplantation of two kinds of cell (human fetal hepatocytes and syngeneic CD34⁺ haematopoietic stem cells (HSCs) or miss-matched human adult hepatocytes and HSCs). When the two kinds of cells are simultaneously transplanted to the mice, the order of transplantation of the two types of cell and the mutual repulsion of the different origins cells need to be considered. If the transplantation sequence, time interval and cell number ratio of the two types of cell are improper, it would easily result in low transplantation efficiency, i.e., the two types of cell are difficult to colonize in the mice at the same time. Meanwhile, the present invention develop a novel dual humanized murine model with efficient chimerism of human liver cells and human immune cell lineages only using a single transplantation of stem cell, and can overcome the limitation existing in the current humanized murine model. Moreover, a single type of stem cell transplantation has a higher efficiency, and there are no rejections of the hepatocytes and immune cells simultaneously differentiated in the murine. The dual-humanized murine model obtained by a single type of stem cell transplantation is more stable. This is not achievable by the existing technique of constructing humanized murine model, and cannot be derived by one skilled in the art based on existing knowledge.

In terms of the selection of the strains of the experimental murine, the existing technique for constructing the humanized murine model only uses immunodeficient mice, and is not applicable for normal immune mice. Meanwhile, the strains of experimental murine used in the present invention are in a broad range, including normal immune mice, immunodeficient mice, immune normal rat, or immunodeficient rat. Since the transplanted cells (such as CD34⁺ cells) used in the existing technique for constructing the humanized murine model are not immunological tolerant, mice with normal immune system will immunologically reject CD34⁺ cells. That is, the immune systems of the mice clear these transplanted cells. Therefore, the existing technique for constructing humanized murine model can only select mice with deficient immune system and cannot select normal immune mice. Meanwhile, the human stem cells used in the present invention have immunological tolerance and no rejection reaction. After transplanting these cells into normal murine, they coexist with the murine immune system and are not cleared, so that it can select normal immune murine which are more readily available.

In order to elucidate the biological characteristics and the specific mechanism of viral morbidity of hepatotropic virus, the present invention has studied the biochemical indicators, immunohistochemistry, gene expression levels, proteomics, and various other aspects. It was found that by inducing severe liver damage and transplanting human stem cells, the human-derived hepatocytes in murine liver have a high chimeric rate of 50-95%, and human-derived immune cells may be continuously isolated from murine organs such as spleen, blood, liver, and bone marrow, thereby forming a murine model of humanized liver and immune cell. The humanized murines are then infected with various types of hepatotropic viruses to form a humanized murine model of hepatotropic viral infection. Such a model may study the entire life cycle of the hepatotropic virus, and the immune response between the hepatotropic viral infection and the humanized immune system formed by transdifferentiation. Liver damage, chronic hepatitis B, liver fibrosis and liver cirrhosis occur successively after 10 to 50 weeks the humanized murines are infected by the hepatotropic viruses, and hepatocellular carcinoma gradually emerges eventually. The humanized murine model is in line with the natural history of hepatotropic viral infection in human, and the development and outcome of viral hepatitis. In addition to the study of the biological characteristics of hepatotropic viruses, the pathogenesis of viral hepatitis, and the research, development and screening of new anti-hepatotropic viral drugs, models of liver cirrhosis, hepatocellular carcinoma, etc. that are more in line with the developmental history of human diseases may also be achieved.

In addition to the construction of a model for studying hepatotropic viral infection using the technique for constructing the humanized murine model, the concept of this technical solution may also be used for constructing models of other humanized organs. The technical solution provides a convenient, simple, and readily available humanized model for the study of clinical treatment of liver diseases.

### BRIEF DESCRIPTION OF THE DRAWINGS

FIG. 1 is a schematic diagram of the construction of a bone marrow mesenchymal stem cell humanized FRGS mouse (hBMSC-FRGS mouse) model.

FIG. 2 is a schematic diagram of the construction of an embryonic stem cell humanized uPA mouse (ES-uPA mouse) model.

FIG. 3 is a schematic diagram of the construction of an induced pluripotent stem cell humanized galactosamine-normal mouse (iPS-normal mouse) model.

FIG. 4 is a construction diagram of a bone marrow mesenchymal stem cell humanized normal rat model.

FIG. 5 is a construction diagram of an adipose-derived mesenchymal stem cell humanized normal rat model.

### DETAILED DESCRIPTION OF THE EMBODIMENTS

The present invention discloses a method of constructing a humanized murine model using human stem cells, and discloses a type of humanized murine model for studying viral hepatitis constructed by the method. The technical solution of the present invention is further explained below:

### I. Obtaining Human Stem Cells

1. Isolating and culturing human stem cells
   1) Obtaining purified human stem cells.
   2) Culturing and subculturing the stem cells.
   3) Incubating in an incubator at 20° C. to 40° C., 2% to 10% CO₂.
2. Obtaining commercialized isolated or frozen human stem cells or cell lines.
   II. Transplanting Stem Cells into Murines with Liver Damage
1. Obtaining different strains of test murine. Examples of test murine include normal mouse, immunodeficient mouse, normal rat, and immunodeficient rat.
2. Establishing a murine model of liver damage by administering liver-damaging drugs through intraperitoneal injection, intramuscular injection, peripheral intravenous injection, oral administration, or gastric administration, or by partial hepatic resection by surgery.
3. Transplanting 1×10⁴⁻⁸ stem cells by means of peripheral intravenous injection, portal vein injection, spleen injection, or liver injection.

### III. HBV Infected Humanized Murine

Injecting each murine with various types of hepatotropic viruses through peripheral intravenous injection, subcutaneous injection, intramuscular injection, or intraperitoneal injection.

### Definitions

“Humanized chimeric murine” refers to a murine model carrying a human-derived cell, tissue or organ in vivo, and is a chimera formed of a transplanted human cell or tissue and the murine. It can better simulate the characteristics of the human body in some aspects, and thus is a more ideal animal model. The “human liver and immune cell dual-humanized murine model” in the present invention refers to a murine with a human immune system and hepatocytes established in vivo. Since HBV adhesion and viral DNA transcription and replication are highly species-specific to the host (only infecting humans, apes/chimpanzees), there is still no ideal animal model to mimic the biological processes of human HBV infection. With the development of transgenic technology, HBV transgenic mice and adenovirus-mediated HBV-infected mice have played a positive role in studying HBV infection process. However, these mice models have some limitations, since they lack cccDNA and HBV is integrated into the chromosome of the host, there is no process of binding to the target cell receptor, which is completely different from the natural human HBV infection. In the previous studies using human primary hepatocyte transplantation to establish humanized chimeric model, only chimera of human hepatocytes is achieved in the murine in vivo, and chimera of immune cells is lacking. Meanwhile, the occurrence and development of HBV infection in relation to immune cells and liver damage caused by HBV infection are essential. Liver damage in patients with hepatitis B is not a direct result of viral replication in the liver, but is a reaction mechanism caused by the immune response of the body to the expressed products of HBV. With the development of stem cell theory and technology, the establishment of human liver and immune cell dual-humanized murine model established by stem cell transplantation is considered to be a promising animal model for simulating the natural infection of human HBV, the pathogenesis of hepatitis B cirrhosis and the animal model of hepatitis B-associated hepatocellular carcinoma.

“Normal immune murine” refers to a mouse or rat with an intact immune system, including inbred mice (BALB/c mice, C3H/He mice, C57BL mice, DBA mice, etc.), and outbred mice (KM mice, ICR mice, NIH mice, CFW mice, LACA mice), SD rats, and Wistar rats. There are many strains of these murine, which are readily available, easy to raise, fast to breed, low in cost (about 3 U.S. dollar each), and have a high survival rate, allowing the dual-humanized murine of the present invention to be more widely applied in the study of hepatitis and related diseases (liver fibrosis, liver cirrhosis and hepatocellular carcinoma).

“Immuno-deficient mice” refers to the mice with deficient immunological function caused by development, differentiation, and metabolic abnormalities of the immune-active cells due to damage to any link or component of the immune system caused by congenital hypoplasia or various acquired factors. Commonly used mice mainly include Nude mice, SCID mice, NOD/SCID mice, NSB mice, NSG mice, NOG mice, FRGS mice and the like.

“uPA model” refers to the albumin-urokinase plasminogen activator (Alb-uPA) transgenic mouse, which was the earliest reported humanized liver model. It is usually crossed with SCID mouse to obtain immunodeficient uPA/SCID mouse. The transfer of uPA causes spontaneous death of hepatocytes, thereby providing a possibility for the construction of humanized liver. At the same time, there are also shortcomings. Firstly, the neonatal transgenic uPA mice have a very high mortality rate, are extremely difficult to breed, prone to bleeding, renal dysfunction, etc., and it is difficult to obtain a large number of recipient mice. Secondly, the transplantation window of the uPA/SCID model is small. That is, cell transplantation surgery must be completed within two weeks of age of the mice, which is very difficult in terms of technical operation.

“FRGS model” refers to the highly immunedeficient FRGS mouse obtained from hybridization of FAH gene-deficient mouse to Rag2^{-/-} IL2Rγ^{-/-} SCID mouse. FAH is an enzyme in the tyrosine metabolic pathway, and its deletion will cause the tyrosine metabolism to transfer into the branch pathway. Metabolic toxicants accumulate in the hepatocytes, thereby causing death of hepatocytes. Meanwhile, long-term consumption of 2-nitro-4-trifluorotoluene-1,3-cyclohexanedione (NTBC) in FAH-deficient mice can eliminate the accumulation of tyrosine intermediate metabolic toxicants, thereby being very good in preventing the occurrence of liver damage. Generally, through regular withdrawal of NTBC, the liver can be slowly and chronically damaged, providing an effective growth space for the cell transplantation.

“Human stem cells” refers to a type of human pluripotent cell with self-replication ability. Under certain conditions, they can differentiate into multiple functional cells. According to the developmental stage at which the stem cells are, they can be classified into embryonic stem cells (ES cells) and adult stem cells. According to the developmental potential of stem cells, they can be divided into three categories: totipotent stem cells (TSC), pluripotent stem cells, and unipotent stem cells (specific stem cells). Stem cells are a type of under-differentiated, immature cell that have the potential to regenerate various tissues, organs and the human body. Human bone marrow mesenchymal stem cells (hBMSCs) are a type of stem cell that are positive for the expression of cell surface markers CD73, CD90 and CD105, but lack the expression of surface markers CD11b, CD14, CD19, CD34, CD45, CD79a and HLA-DR. They have strong proliferative capacity and multipotential differentiation, and have the ability to transdifferentiate into multiple lineages such as muscle cells, hepatocytes, osteoblasts, fat cells, chondrocytes, stromal cells, etc. under the suitable in vivo or in vitro environment. They are immunotolerant and will not be rejected by the murine immune system when transplanted into the murine. Human embryonic stem cells (hES) are a type of primitive pluripotent stem cell derived from human blastocyst inner cell mass and obtained from isolation and culturing in vitro, and can be induced to produce various specialized cells and tissues by directed differentiation, which are used to repair or replace tissues and organs that have lost function. Human induced pluripotent stem cells (iPSCs) are a type of cell which are similar to embryonic stem cells and embryonic APSC pluripotent cells, obtained by transfecting a combination of four transcription factors (Oct4, Sox2, Klf4 and c-Myc) into differentiated somatic cells using a viral vector in order to reprogram them. iPSCs have the potential of self-renewal and multi-differentiation. Human adipose-derived mesenchymal stem cells are a type of fibroblast-like mesenchymal cell present in human body fat. They are small in size, low in degree of differentiation, have better tolerance for trauma and hypoxia than fat cells, and have multi-differentiation potential. Furthermore, they are easy to obtain and rich in source relative to other stem cells. These stem cells are important sources of transplanted cells for constructing the humanized murine.

“Liver failure” generally includes acute liver failure (ALF), subacute liver failure (SALF), chronic acute (subacute) liver failure (ACLF), and chronic liver failure (CLF) based on histopathological features and the speed of disease development. In terms of the types of modeling, there are drug-induced liver failure model and surgical liver failure. The surgical liver failure model mainly includes partial hepatectomy model and acute liver ischemia model. The drug-induced liver failure model mainly includes acetaminophen model, aminogalactose model, thioacetamide (TAA) model, and carbon tetrachloride (CCl₄) model. In addition, anti-Fas antibody (JO2), combined with Fas ligand or specific antibody (such as JO2 mAb), activates the APO-1/Fas pathway and specifically induces apoptosis in mice, which can construct the JO2 mouse liver failure model.

The advantage of the present study is the construction of a liver and immune cell dual-humanized murine model from immune normal mice or immune normal rats simply by transplanting a single type of stem cell. This technique can differentiate human functional hepatocytes and immune cell lineages using a single transplantation of stem cell, which can avoid the problems of improper cell transplantation order, time interval, and cell number ratio, and the easily resulted low transplantation efficiency. The present technique can use immune normal murine, which are simple to breed, fast to breed, low in cost, high in survival rate, and have a wide selection and low requirements for experimental animal strains, which can greatly reduce the use cost of experimental animals and make the contruction of humanized model more simple and convenient, and can be more widely applied in research.

### EXAMPLES

The technical solution of the present invention will be further described below in accordance with the drawings, specific embodiments and comparative examples. Through injecting various types of human-derived stem cell into murine with liver damage, the present invention has obtained different humanized murine models for studying the infection mechanism of hepatotropic viruses, and the occurrence, development mechanism, outcome and treatment of hepatotropic viral infection.

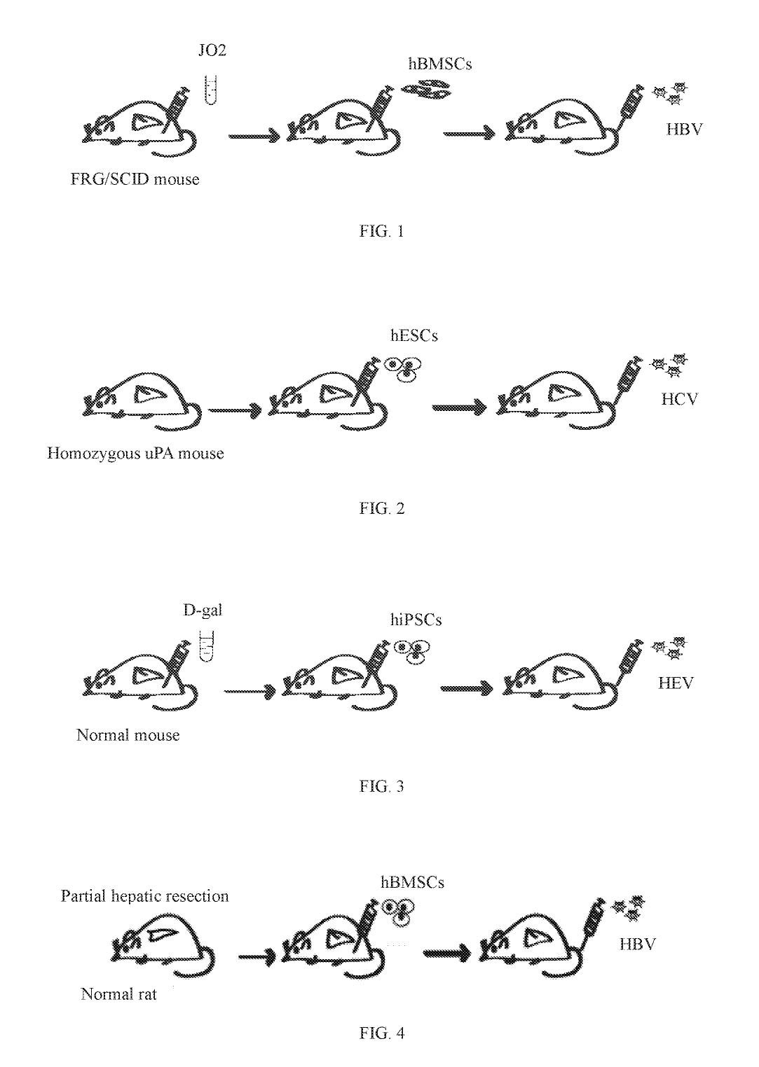

Example 1: FIG. 1 is a schematic diagram of the construction of a bone marrow mesenchymal stem cell humanized FRGS mouse model. Human bone marrow mesenchymal stem cells (hBMSCs) were transplanted into FRGS mice with fulminant hepatic failure to establish a human liver and immune cell dual-humanized mouse model.
1. Human bone marrow mesenchymal stem cells (hBMSCs) were cultured in DMEM culture medium containing 10% fetal bovine serum.
2. The amount of drug 2-(2-nitro-4-trifluoromethylbenzyl)-cyclohexane-1,3-dione (NTBC) was gradually reduced and 0.2 mg/kg of anti-Fas antibody (JO2) was injected into FRGS mice to establish a mouse model of liver failure.
3. 500 ul of 1×10⁶ hBMSCs were injected through portal vein injection.
4. JO2 injection was continued for two, five and eight days after the transplantation.
5. 1×10⁶ of each of the sub-genotypes A, B, C and D of hepatitis B virus were injected into each mouse through tail vein injection.
6. At one week, two weeks and four weeks after the injection of the hepatitis B virus, the viral load of the hepatitis B virus and the status of the liver function were measured every four weeks thereafter to confirm that the model was successfully established.

FRGS mice were used in FIG. 1. Firstly, the chemical drug NTBC retreating on FRGS mice was used for establishing fulminant hepatic failure. Then, the human bone marrow mesenchymal stem cells were transplanted and differentiated into human hepatocytes and immune cells. Lastly, hepatitis B viruses were injected to establish a humanized mouse model of chronic hepatitis B.

Example 2: FIG. 2 is a schematic diagram of the construction of an embryonic stem cell humanized uPA mouse model. Human embryonic stem cell line was transplanted into homozygous uPA mice to establish an embryonic stem cell humanized uPA mouse model.
1. Human embryonic stem cells were cultured in DMEM culture medium containing 10% fetal bovine serum.
2. Homozygous uPA/SCID mouse model was obtained.
3. 500 ul of 1×10⁶ human embryonic stem cells were injected through spleen injection at eight weeks after birth.
4. 1×10⁷ hepatitis C viruses were injected into each mouse through tail vein injection.
5. At one week, two weeks and four weeks after the injection of the hepatitis C virus, the viral load and the status of the liver function were measured every four weeks thereafter to confirm that the model was successfully established.

uPA mice were used in FIG. 2. Liver damage is spontaneously developed in uPA mice. The human embryonic stem cells were then transplanted and differentiated into human hepatocytes and immune cells. Lastly, hepatitis C viruses were injected to establish a humanized mouse model of chronic hepatitis C.

Example 3: FIG. 3 is a schematic diagram of the construction of an induced pluripotent stem cell humanized galactosamine normal mouse model. Human induced pluripotent stem cells (hiPSCs) were injected into immunologically normal mice with fulminant hepatic failure to establish an induced pluripotent stem cell humanized normal mouse model.
1. Certain transcription factors were introduced into animal or human somatic cells by gene transfection technique, causing the somatic cells to be directly reconstituted to form pluripotent stem cells. The pluripotent stem cells were cultured in DMEM culture medium containing 10% fetal bovine serum.
2. 1.5 g/kg galactosamine was injected into the abdominal cavity of each mouse to establish a mouse model of liver failure.
3. 500 ul of 1×10⁶ human induced pluripotent stem cells were injected through liver injection.
4. 1×10⁵ hepatitis E viruses were injected into each mouse through tail vein injection.
5. At one week, two weeks and four weeks after the injection of the hepatitis E virus, the viral load and the status of the liver function were measured every four weeks thereafter to confirm that the model was successfully established.

Mice were used in FIG. 3. Firstly, the chemical drug galactosamine was used for establishing fulminant hepatic failure. Then, the human induced pluripotent stem cells were transplanted and differentiated into human hepatocytes and immune cells. Lastly, the hepatitis E viruses were injected to establish a humanized mouse model of chronic hepatitis E.

Example 4: FIG. 4 is a schematic diagram of the construction of a bone marrow mesenchymal stem cell humanized normal rat model. Human bone marrow mesenchymal stem cells (hBMSCs) were injected into normal rats with acute liver damage to establish a bone marrow mesenchymal stem cell humanized normal rat model.
1. Purified human bone marrow mononuclear cells were isolated from normal human bone marrow using lymphocyte separation medium and cultured in DMEM culture medium containing 10% fetal bovine serum to obtain human bone marrow mesenchymal stem cells (hBMSCs).
2. The normal rats were subject to 50% hepatectomy to establish a rat model of acute liver damage.
3. 500 ul of 1×10⁶ hBMSCs were injected through portal vein injection.
4. 1×10⁶ of each of the sub-genotypes A, B, C and D of hepatitis B virus were injected into each rat through tail vein injection.
5. At one week, two weeks and four weeks after the injection of the hepatitis B virus, the viral load and the status of the liver function were measured every four weeks thereafter to confirm that the model was successfully established.

Normal rats were used in FIG. 4. Firstly, 50% hepatic resection by surgery was used for establishing acute liver damage. Then, the human bone marrow mesenchymal stem cells were transplanted and differentiated into human hepatocytes and immune cells. Lastly, hepatitis B viruses were injected to establish a humanized rat model of chronic hepatitis B.

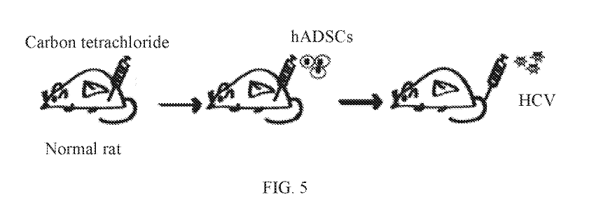

Example 5: FIG. 5 is a schematic diagram of the construction of a human adipose-derived mesenchymal stem cell humanized normal rat model. Human adipose-derived mesenchymal stem cells (hADSCs) were injected into normal rat with liver damage to establish an adipose-derived mesenchymal stem cell humanized normal rat model.
1. Purified human adipose-derived mesenchymal stem cells were isolated from normal human adipose tissue and cultured in DMEM culture medium containing 10% fetal bovine serum to obtain human adipose-derived mesenchymal stem cells (hADSCs).
2. Carbon tetrachloride (0.5 ml/100 g) was injected into normal rats through intraperitoneal injection to establish a rat model of acute liver damage.
3. 1000 ul of 5×10⁶ hADSCs were injected through spleen injection.
4. 1×10⁶ hepatitis C viruses were injected into the abdomen cavity of each rat.
5. At one week, two weeks and four weeks after the injection of the hepatitis C virus, the viral load and the status of the liver function were measured every four weeks thereafter to confirm that the model was successfully established.

Normal rats were used in FIG. 5. Firstly, the chemical drug carbon tetrachloride was used for establishing acute liver damage. Then, the human adipose-derived mesenchymal stem cells were transplanted and differentiated into human hepatocytes and immune cells. Lastly, hepatitis C viruses were injected to establish a humanized rat model of chronic hepatitis C.

The above examples are merely the preferred embodiments of the present invention, and the present invention is not limited to the above embodiments. Other improvements and changes directly deduced or conceived of by one skilled in the art without departing from the gist and concept of the present invention should be considered as being included in the scope protected by the present invention.

What is claimed is:

1. A method of constructing a humanized murine model using human stem cells, comprising the following steps:
(a) obtaining the human stem cells;
(b) transplanting the human stem cells into a murine with liver damage; and
(c) obtaining a hepatotropic virus infected humanized murine.

2. The method according to claim 1, wherein the human stem cells are isolated and cultured human stem cells, or commercialized isolated or frozen human stem cells or cell line.

3. The method according to claim 2, wherein the isolated and cultured human stem cells are obtained by the following steps:
(i) obtaining purified human stem cells;
(ii) culturing and subculturing the purified human stem cells;
(iii) incubating the cultured, subcultured, purified human stem cells obtained from the step (iv) in an incubator at 20° C. to 40° C., 2% to 10% CO₂.

4. The method according to claim 1, wherein the step (b) comprises the following steps:
(b1) obtaining different strains of test murine;
(b2) establishing the murine with liver damage by administering a liver-damaging drug, or applying a partial hepatic resection by surgery;
(3) transplanting 1×10⁴⁻⁸ of the human stem cells into the murine with liver damage.

5. The method according to claim 4, wherein the step (b) further comprises the following step after the step (b3):
(b4) administering the liver-damaging drug in several times.

6. The method according to claim 4, wherein the test murine is selected from the group consisting of normal mouse, immunodeficient mouse, normal rat, and immunodeficient rat;
wherein the liver damage comprises any one of the following: acute liver damage, chronic liver damage, acute liver failure, subacute liver failure, and chronic liver failure.

7. The method according to claim 4, wherein in the step (b2), the liver-damaging drug is administered through intraperitoneal injection, intramuscular injection, peripheral intravenous injection, oral administration, or gastric administration.

8. The method according to claim 4, wherein in the step (b3), the 1×10⁴⁻⁸ of the human stem cells are transplanted through peripheral intravenous injection, portal vein injection, spleen injection, or liver injection.

9. The method according to claim 1, wherein the step (c) comprises injecting each of the murine with liver damage with various types of hepatotropic viruses through peripheral intravenous injection, subcutaneous injection, intramuscular injection, or intraperitoneal injection.

10. The method according to claim 1, further comprising the following step after the step (c):
(d) measuring viral load at least once within 3-30 days after the murine with liver damage is infected to confirm that a viral hepatitis model is successfully established; after measuring the viral load for once within 3-30 days after the murine with liver damage is infected, measuring the viral load again every 4 weeks thereafter in several times to confirm that the humanized murine model is successfully established.
